(19) United States
(12) Patent Application Publication
(10) Pub. No.: US 2020/0069540 A1
(43) Pub. Date: Mar. 5, 2020

(54) COSMETIC FOAM CONTAINING WAXES AND POLYACRYLIC ACID POLYMERS

(71) Applicant: BEIERSDORF AG, Hamburg (DE)

(72) Inventors: Christian DINGLER, Hamburg (DE); Kerstin SKUBSCH, Prisdorf (DE); Isabel BALCKE, Hamburg (DE); Svea WISCHHOEFER, Hamburg (DE)

(21) Appl. No.: 16/493,780

(22) PCT Filed: Mar. 20, 2018
(86) PCT No.: PCT/EP2018/056943

§ 371 Date: Sep. 13, 2019

(30) Foreign Application Priority Data
Mar. 21, 2017 (DE) 10 2017 204 709.7

(51) Int. Cl. A61K 8/04; A61K 8/31; A61K 8/34; A61K 8/37; A61K 8/81; A61K 8/92; A61Q 19/00; A61Q 9/02

(52) U.S. Cl. CPC A61K 8/046; A61K 8/31; A61K 8/342; A61K 8/37; A61K 2800/33; A61K 8/922; A61K 8/8147; A61Q 19/00; A61Q 9/02; A61K 8/8152

(57) ABSTRACT

The present invention relates to an emulsifier-free and surfactant-free cosmetic product having a pressure container with a release valve containing at least 8% by weight of one or more waxes and polyacrylic acid polymers. The pressure container further contains at least one propellant that foams and/or discharges the preparation.

The present invention relates to a cosmetic product comprising a pressure container with a release valve comprising at least 8% by weight of one or more waxes and one or more polyacrylic acid polymers, wherein the preparation comprises no surfactants and no emulsifiers. In addition, the pressurized container comprises at least one propellant that foams and/or discharges the cosmetic preparation. The cosmetic product according to the invention enables the provision of a caring foam which is particularly suitable to be applied to wet or moistened skin and then to be rinsed off. The resulting foam has a surprising structural stability and allows the application of a nurturing protective film to the skin.

To appear beautiful and attractive is a need for many humans. A pure and well-groomed skin is often an ideal of beauty. In order to accord to this, a wide variety of different cosmetic and dermatological preparations are used for daily cleansing and care of the skin.

Generally, such cosmetic and dermatological preparations can be classified into two categories. On one side are the “rinse off” preparations which are washed off the skin with water immediately after application. Typical preparations in this category are, for example, shower gels or hair shampoos. On the other side are the so-called “leave on” preparations which remain on the skin after application in order for these to be absorbed. Typical preparation examples are creams, lotions and body milk.

Both “leave on” and “rinse off” preparations can be formulated in the form of emulsions in which oil is dispersed in water or water is dispersed in oil.

If attempts are made to disperse oil in water or water in oil, the problem generally occurs that the dispersed droplets rapidly agglomerate and thus phase separation occurs. In order to prevent this, emulsifiers are used which enable the dispersed droplets to stabilize. In this manner, phase separation can also be prevented long term. Particularly in shower and shampoo preparations, surfactants are present which serve, inter alia, to form foam on application to the skin.

“Rinse off” preparations that can be rinsed off, which are applied to moist or wet skin, such as shower and shampoo preparations, are usually less useful for skin care. On the contrary, the surfactants present allow effective removal of fat, perspiration and impurities which are on the skin.

As an alternative to classical shower gels, which are withdrawn from the storage container in the liquid state and applied to the skin for cleansing the skin, there are a small number of products in which the surfactant-based cleansing preparation is foamed for application to the skin with the aid of propellant gas. In this case, a foam with a mousse texture is obtained, which is not only fun for the user on application to the skin but is also usually perceived as well-distributed on the skin.

The surfactants and emulsifiers present in these products promote foam formation such that structurally stable foams are usually obtained. However, a disadvantage is that such products do not permit adequate care of the skin. For instance, after showering, the skin often feels dry and taut after application. In order to counteract this, it is usually necessary to apply body care lotion after showering.

However, if body care lotions, which are usually formulated as oil-in-water emulsions, are applied directly to wet skin under the shower, there is the disadvantage that no nurturing residue remains on the skin due to the emulsifiers present. Instead, the lipid components present emulsified in the body lotion are kept in solution. Accordingly, application to wet skin in the shower is not very useful. This also applies to body lotions as O/W emulsions foamed with propellant gas.

A particular disadvantage is that omitting or reducing surfactants and emulsifiers results in inadequate foams. Accordingly, particularly surfactant-free and emulsifier-free preparations with a high proportion of nurturing waxes foam only poorly or not at all. Moreover, these products have the disadvantage that they can be poorly rinsed off with water. It is further disadvantageous that the foams obtained rapidly degrade, usually in less than 5 minutes, and therefore have no structural stability. It is also disadvantageous that an inadequate amount of lipids (oil or wax components) is deposited on the skin.

The prior art includes the document EP 1992323 A1, which relates to foamy preparations which exclusively comprise solid emulsifiers. However, the preparations do not comprise any waxes.

EP 2335675 A1 discloses foam formulations comprising a solid emulsifier and are therefore not emulsifier-free. The foams obtained degrade in a few minutes.

None of the aforementioned documents could lead a person skilled in the art to the present invention.

Surprisingly, a cosmetic product providing a structurally stable foam that can be readily washed out has now been found which does not have the disadvantages and problems described.

The invention is a cosmetic product having a pressure container with a release valve comprising
a) a cosmetic preparation—comprising, based on the total weight of the cosmetic preparation—
   (i) a total of 8% by weight to 35% by weight, preferably 9.5% by weight to 25% by weight and especially to 22% by weight of one or more waxes different from fatty alcohols, and
   (ii) one or more polyacrylic acid polymers,
characterized in that the preparation comprises no surfactants and no emulsifiers;
b) at least one propellant that foams and/or discharges the cosmetic preparation.

Also subject matter of the invention is the use of the cosmetic product according to the invention for providing a foam for application to wet skin.

Also subject matter of the invention is the use of the cosmetic product according to the invention for providing a shaving foam. If such a foam is used as a shaving foam, the razor blade exhibits particularly good lubricant properties. In addition, a surprisingly low skin drying is achieved in comparison with shaving foams which are based on surfactants such as soaps. Accordingly, such foams are experienced as pleasant and nurturing.

Also subject matter of the invention is the use of
(i) a total of 8% by weight to 35% by weight, preferably 9.5% by weight to 25% by weight and especially to 22% by weight of one or more waxes different from fatty alcohols, and
(ii) one or more polyacrylic acid polymers

in a cosmetic preparation foamed with propellant gas containing no surfactants and emulsifiers, for providing a foam which, on application to wet skin, deposits a lipid film on the skin, wherein the % by weight figures refer to the total weight of the cosmetic preparation without taking the propellant gas into consideration.

Also subject matter of the invention is the use of
(i) in total 8% by weight to 35% by weight, preferably 9.5% by weight to 25% by weight and especially to 22% by weight of one or more waxes different from fatty alcohols, and
(ii) one or more polyacrylic acid polymers

in a cosmetic preparation foamed with propellant gas containing no surfactants and emulsifiers, for providing a structurally stable foam, wherein the % by weight figures refer to the total weight of the cosmetic preparation without taking the propellant gas into consideration. The foam obtained collapses less quickly after discharging and/or foaming with the propellant, especially after a period of 5 minutes, than foams of the prior art.

Also subject matter of the invention is a method for applying the cosmetic product according to the invention, characterized in that in a first step the cosmetic preparation is withdrawn from the pressure container, in a second step the (foamed) cosmetic preparation is applied to skin moistened with water and in a third step is rinsed off the skin with water.

Also subject matter of the invention is a method for applying the cosmetic product according to the invention, characterized in that in a first step the cosmetic preparation is withdrawn from the pressure container, in a second step the (foamed) cosmetic preparation is applied to hirsute skin moistened with water and in a third step the hair in the skin region to which the (foamed) cosmetic preparation has been applied is shaved with a razor blade.

If the term skin is used in this disclosure, this refers exclusively to human skin.

The cosmetic product according to the invention forms a surprisingly structurally stable foam after spreading which, even after a period of 5 minutes, degrades less than comparable foams of the prior art. It was also surprising that, after spreading the resulting foam on the skin and rinsing off the foam within 5 minutes after application, a residue remains thereon which imparts a nurturing pleasant feel. The residue consists predominantly of lipids. Accordingly, it was surprisingly possible to provide a foam which is applied to wet skin and then is rinsed off with water, and which in this case does not have or reduces the dry skin feel that occurs with foaming products. It was also surprising that the foam according to the invention can be particularly easily distributed on wet skin despite the high wax content. Moreover, the foams obtained according to the invention are surprisingly finely pored.

All percentages by weight listed below (% by weight), unless stated otherwise, refer in each case to the total weight of the cosmetic preparation.

All experiments were carried out under standard conditions unless stated otherwise. The expression “standard conditions” signifies 20° C., 1013 hPa and a relative humidity of 50%.

In the context of the present disclosure, the expression “free of” signifies that the fraction of the respective substance is less than 0.05% by weight. As a result, it is ensured that infiltrations or impurities with these substances are not included as “free of” according to the invention.

In accordance with the invention, fatty alcohols include long-chain primary alcohols having 6 to 22 carbon atoms. The fatty alcohols having 6 to 22 carbon atoms also by definition do not include the surfactants and emulsifiers.

In the context of the present invention, waxes are understood to mean natural and synthetic substances which at 20° C. are kneadable, solid to brittle hard, coarse to finely crystalline, transparent to opaque but not glassy; melt without decomposition above 40° C., are of relatively low viscosity slightly above the melting point and are not thread-forming, have highly temperature-dependent consistency and solubility, and can be polished under slight pressure.

In accordance with the invention, the wax(es) different from the fatty alcohols are selected from natural and/or synthetic waxes.

The naturally occurring waxes which can be used in the usual way in cosmetic products are selected from natural plant waxes, for example carnauba wax, candelilla wax, sunflower wax and/or fruit waxes (for example apple wax or citrus wax). Further natural waxes in accordance with the invention are animal waxes such as beeswax, wool wax, spermaceti and preen gland wax.

In accordance with the invention, synthetic waxes include both waxes that are produced fully synthetically and chemically modified waxes. Accordingly, synthetic waxes which may be present in the preparation according to the invention are, for example, hard paraffin or cera microcristallina. Here, cera microcristallina is a generic term for alternative names such as microcrystalline wax, microcrystalline waxes or mineral wax.

Cera microcristallina is a complex combination of long, branched hydrocarbon chains which are obtained from residual oils by solvent crystallization. Cera microcristallina consists predominantly of saturated straight-chain and branched hydrocarbon chains, predominantly having more than 35 carbon atoms. Cera microcristallina was formally referred to as Vaseline®, which is nowadays offered as a brand name of ChesePorough Ponds.

If cera microcristallina is used as wax in accordance with the invention, this is advantageously characterized in that no polycyclic aromatics or sulfur-containing compounds are present. If these specifications are met, cera microcristallina has no allergic potential by reason of its chemical neutrality.

Further synthetic waxes in accordance with the invention are esters of long-chain fatty acids and long-chain fatty alcohols, wherein it is advantageous if the fatty acids have 15 to 36 carbon atoms and the fatty alcohols have 15 to 36 carbon atoms. Examples in accordance with the invention are, inter alia, cetyl stearate or cetyl palmitate.

Furthermore, chemically modified waxes include hardened or hydrogenated vegetable oils. Typical examples in accordance with the invention are known under the INCI names Hydrogenated Castor Oil, Hydrogenated Vegetable Oil, Hydrogenated Avocado Oil, Hydrogenated Cotton Seed Oil, Hydrogenated Rapeseed Oil, Hydrogenated Sunflower Oil, Hydrogenated Palm Oil, Hydrogenated Jojoba Oil, Hydrogenated Soybean Oil or Hydrogenated Coconut Oil.

It is preferable in accordance with the invention if at least one wax is present in the cosmetic preparation selected from the group cera microcristallina, hydrogenated jojoba oil and/or hydrogenated vegetable oil.

The maximum proportion of the waxes present in the cosmetic preparation is 35% by weight, preferably 25% by weight and particularly preferably 22% by weight, based on the total weight of the cosmetic preparation.

In accordance with the invention, the term polyacrylic acid polymer is understood to mean polymers used in cosmetics which are polymerized from a monomer mixture comprising acrylic acid and/or methacrylic acid (a.). An example of polyacrylic acid polymers which consist of polyacrylic acid and optionally small amounts of a crosslinking substance are referred to as carbomers according to INCI nomenclature. Often used as crosslinking substance are, for example, pentaerythritol allyl ether, allyl ether of propylene, or allyl ethers of sucrose (for example known under the CAS number: 68784-14-5).

Carbomers have the property that these form anionic charges on the carboxyl groups in aqueous solution dependent on the pH. This results in repulsion of the deprotonated groups and stretching of the polymer chains. Due to this effect, a linear colloidal structure is formed which can store water. Due to the storage of water, a gel structure is formed which leads to an increase in the viscosity of the solution. The deprotonation of the carboxyl groups is dependent on the pH such that dissolution of the gel network may occur that depends on this. Polyacrylic acid polymers according to the invention which are commercially available under the INCI name carbomer are the commercial products with the name Carbopol and the additive EDT 2050, 934, 940, 941, 980, 981, 2984, 5984, 934P and Ultrez 10, which can all be obtained from Lubrizol.

A further group of polyacrylic acid polymers according to the invention are characterized in that these are polymerized from a monomer mixture which, in addition to the short-chain monomers acrylic acid and/or methacrylic acid (a.), additionally comprise C₁- to C₄₀-alkyl acrylates and/or C₁- to C₄₀-alkyl methacrylates (b.) as monomer constituents. These polymers can also be present as copolymer or crosslinked as crosspolymer. Typical examples of these polymers are known under the INCI names Acrylates/C12-22 Alkyl Methacrylate Copolymer, Acrylates/C10-30 Alkyl Acrylate Crosspolymer and Acrylates

Copolymer. Acrylates/C12-22 Alkyl Methacrylate Copolymer is obtainable under the trade name Soltex OPT from DOW Chemicals. The polymer Acrylates/C10-30 Alkyl Acrylate Crosspolymer can be obtained, inter alia, under the trade names Pemulen TR-1 Polymer, Pemulen TR-2 Polymer, Carbopol Ultrez 20 Polymer, Carbopol Ultrez 21 Polymer, Carbopol 1342 Polymer, Carbopol 1328 Polymer or Carbopol ETD 2020 Polymer from Lubrizol. The polymer Acrylates Copolymer is commercially available under the trade names Aculyn 33 from DOW Chemicals or Carbopol Aqua SF-1 from Lubrizol.

Further polyacrylic acid polymers according to the invention additionally comprise as monomer constituents ethylenically unsaturated hydrophobically modified monomers (c.) which may be selected from the group polyethoxylated cetyl (meth)acrylate, polyethoxylated cetearyl (meth)acrylate, polyethoxylated stearyl (meth)acrylate, polyethoxylated arachidyl (meth)acrylate, polyethoxylated behenyl methacrylate, polyethoxylated cerotyl (meth)acrylate, polyethoxylated montanyl (meth)acrylate, polyethoxylated melissyl (meth)acrylate, polyethoxylated lacceryl (meth)acrylate, polyethoxylated nonylphenol (meth)acrylate, polyethoxylated tristyrylphenol (meth)acrylate, polyethoxylated palmyl itaconate, polyethoxylated stearyl itaconate, polyethoxylated behenyl itaconate, polyethoxylated palmyl maleate, polyethoxylated stearyl maleate, polyethoxylated behenyl maleate, polyethoxylated palmyl allyl ether, polyethoxylated stearyl allyl ether, polyethoxylated behenyl allyl ether, wherein the polyethoxylated portion of the monomers above comprises 15 to 60 repeating ethylene oxide units. Typical examples of such polymers according to the invention are known under the INCI name Acrylates/Beheneth-25 Methacrylate Copolymer (for example Aculyn 28 from DOW Chemicals) or Acrylates/Steareth-20 Methacrylate Copolymer (for example Aculyn 22 from DOW Chemicals).

Preferred polyacrylic acid polymers are the substances known under the INCI names Carbomer and/or Acrylates/C10-30 Alkyl Acrylate Crosspolymer. In this case, it is preferred if Acrylates/C10-30 Alkyl Acrylate Crosspolymer is present in the cosmetic preparation.

Advantageous in accordance with the invention are the polyacrylic acid polymers characterized in that these polyacrylic acid polymers as a 0.2 percent solution in water have a viscosity after neutralization of 1500 to 10 000 mPa·s at a temperature of 25° C. The measurement is effected using a Brookfield RV Viscosimeter, RV spindle 4 or RV spindle 5 (selection depending on the viscosity range), in accordance with Lubrizol Test Procedure 430-I, Edition August, 2010. Commercial products having a viscosity in the defined range are, inter alia, Carbopol 981 from Lubrizol, which has the INCI name Carbomer, and Pemulen TR-1 from Lubrizol, which has the INCI name Acrylates/C10-30 Alkyl Acrylate Crosspolymer.

Advantageously, the total fraction of polyacrylic acid polymers in the cosmetic preparation is 0.02 to 2% by weight, preferably 0.05 to 1% by weight and particularly preferably 0.1 to 0.5% by weight, based on the total weight of the cosmetic preparation.

In accordance with the invention, the cosmetic preparation does not comprise any surfactants.

Surfactants are substances which lower the surface tension of a liquid or the interfacial tension between two phases and enable or support the formation of dispersions. Surfactants allow two liquids that are technically immiscible with each other, such as oil and water for example, to be able to be dispersed.

Furthermore, surfactants are described as amphiphilic substances which can dissolve organic non-polar substances in water. Due to their specific molecular construction having at least one hydrophilic and one hydrophobic molecular moiety, they ensure a lowering of the surface tension of the water, wetting of the skin, facilitation of soil removal and dissolution, easy rinsing off and—as desired—for foam regulation.

The hydrophilic moieties of a surfactant molecule are usually polar functional groups, for example —COO⁻, —OSO₃²⁻, —SO₃⁻, whereas the hydrophobic moieties are generally non-polar hydrocarbon radicals. Surfactants are generally classified according to type and charge of the hydrophilic molecular moiety. For this purpose, four groups can be distinguished:
anionic surfactants,
cationic surfactants,
amphoteric surfactants and
non-ionic surfactants.

Anionic surfactants generally have carboxylate, sulfate or sulfonate groups as functional groups. In aqueous solution, they form negatively charged organic ions in acidic or neutral medium. Cationic surfactants are almost exclusively characterized by the presence of a quaternary ammonium group. In aqueous solution, they form positively charged organic ions in acidic or neutral medium. Amphoteric surfactants comprise both anionic and cationic groups and accordingly behave as anionic or cationic surfactants in aqueous solution depending on the pH. In strongly acidic medium, they have a positive charge and in alkaline medium a negative charge.

Furthermore, detersive substances are known, such as cationic surfactants for example, especially quaternary ammonium compounds. A detersive substance is used in detergents, rinse aids, shampoos, shower gels and refer to the part of the formulation which influences the washing or cleaning performance. Detersive substances increase the “solubility” of grease and soil particles in water, which adhere to the laundry or on the body. Moreover, such surfactants promote foam formation. The surfactants can be of natural or synthetic origin.

In addition, the cosmetic preparation according to the invention is characterized in that these do not comprise any emulsifiers.

Emulsifiers allow two liquids that are immiscible with each other (for example oil in water) to be able to mix to give an emulsion. Owing to the amphiphilic character, they penetrate the oil with their fat-soluble moiety. By means of the hydrophilic moiety, the oil droplets now formed by stirring in the aqueous environment are dispersed. Emulsifiers primarily have no washing-active surfactant character. Moreover, the use of emulsifiers can increase the foam formation of a preparation, as well as improve the structural stability of the foam.

Against this background, it is particularly surprising that the preparation according to the invention can be foamed and, as described, a particularly advantageous foam is obtained.

The cosmetic preparation of the present invention is present in a pressure container in accordance with the invention. In said container, in addition to the preparation according to the invention, is a propellant.

The propellants referred to in accordance with the invention are the gases of the group propane, butane, isobutane, pentane, isopentane, dimethyl ether, nitrogen and carbon dioxide. In this case, it is advantageous in accordance with the invention if butane, isobutane and/or propane are selected as propellant. Not to be used are fluorochlorohydrocarbons which damage the environment. The use of pentane or isopentane is less preferred since these slowly evaporate after release from the packaging material owing to the low boiling point and thus the foam formation takes place more slowly. In addition, a less voluminous foam is obtained. It is particularly advantageous if butane, isobutane and/or propane are present and preferably no further propellants are present. In the case of exclusive use of butane, isobutane and/or propane, a more consistent foam based on pore size is obtained.

The mixing ratio of these gases is varied in each case depending on the pressure rating, for example:

Pressure rating 2.7 bar: 60% by weight butane, 20% by weight propane and 20% by weight isobutane;

Pressure rating 3.0 bar: 5.3% by weight butane, 15.3% by weight propane and 79.4% by weight isobutane;

Pressure rating 3.5 bar: 5% by weight butane, 23% by weight propane and 72% by weight isobutane;

based in each case on the total weight of the entire propellants.

Preferred in accordance with the invention are pressure ratings of 2.7 bar, 3.0 bar and 3.5 bar. Particular preference is given to pressure ratings of 3.0 bar and 3.5 bar.

The term “pressure rating” refers to the pressure under which at 20° C. the cosmetic preparation according to the invention is stored or filled in the pressure container using the corresponding propellant gas or propellant gas mixture.

It is advantageous according to the invention if in the pressure container are 90% by weight to 98% by weight cosmetic preparation and 2% by weight to 10% by weight propellant gas or propellant gas mixture, based on the total weight of all liquids and gases present in the pressure container. In this case, it is preferred in accordance with the invention if the proportion of cosmetic preparation according to the invention is 92% by weight to 96% by weight and the proportion of propellant gas or propellant gas mixture is 4% by weight to 8% by weight, based on the total weight of all liquids and gases present in the pressure container.

In addition, it is advantageous in accordance with the invention if the cosmetic preparation is characterized in that this has at least one melting range between 35° C. and 42° C. according to DSC. A further melting range or more melting ranges can lie outside this temperature interval.

DSC (differential scanning calorimetry) is a thermal process for measuring the heat emitted/absorbed by a sample in isothermal mode of operation, heating or cooling (see DIN 53765:1994-03, DIN 51007:1994-06, ASTM E 474-80, ASTM D 3418-15). DSC is a comparative measuring method which enables the determination of amounts of heat transferred in physical and chemical processes. If a material changes its physical state, such as melting or conversion of one crystal form into another or if it reacts chemically, heat in this case is emitted or absorbed. These amounts of heat are measurable quantitatively by DSC. The method proceeds cyclically such that after the first heating curve, a defined cooling takes place and then the sample is heated again in the specified temperature range. Thus, the information obtained is two-fold: in the first heating curve, all thermal effects can be seen including thermal history. In the second heating curve, the thermal history has been eliminated and the pure thermal characteristics of the sample under defined cooling conditions can be evaluated. The melting range of the cosmetic preparation between 35° C. and 42° C. according to DSC is in the range determined in the first heating curve.

If the cosmetic preparation has at least one melting range between 35° C. and 42° C. according to DSC, it has been shown that the preparation attaches particularly effectively to wet skin and leaves a wax film thereon after rinsing off the preparation. Therefore, the deposition of lipids is particularly effective in the presence of at least one melting range between 35° C. and 42° C.

Furthermore, the cosmetic preparation according to the invention is advantageously characterized in that said cosmetic preparation comprises one or more fatty alcohols having 6 to 22 carbon atoms at a total fraction of 0.1 to 15% by weight, based on the total weight of the cosmetic preparation. It is preferred if the total fraction of fatty alcohols having 6 to 22 carbon atoms is from 2% by weight to 9% by weight, based on the total weight of the cosmetic preparation.

The fatty alcohols having 6 to 22 carbon atoms are preferably selected from the group of linear fatty alcohols, especially myristyl alcohol (C₁₄H₃₀O), cetyl alcohol (C₁₆H₃₄O), stearyl alcohol (C₁₈H₃₈O) and mixtures of these such as cetearyl alcohol for example. If mixtures of fatty alcohols are used, particularly myristyl alcohol, cetyl alcohol and stearyl alcohol are therefore present in the preparation according to the invention. In this case it is particularly preferred if the proportion by weight of fatty alcohols having 14 carbon atoms in relation to the proportions by weight of fatty alcohols having more than 14 carbon atoms is from 1:3 to 1:8.

Furthermore, it is advantageous in accordance with the invention if the cosmetic preparation additionally comprises one or more oils which can contribute to care of the skin. In the context of the present disclosure, oils are defined as liquid organic compounds that are insoluble in water at 20° C. and 1013 HPa. Insoluble in water signifies that the solubility of the respective substance at 20° C. is less than 0.1 g/100 g.

In accordance with the invention, the cosmetic preparation may comprise, inter alia, one or more hydrocarbon oils selected from the group mineral oil, paraffin oil and isoparaff in oil.

If, for example, paraffin oil and/or isoparaff in oil is present in the cosmetic preparation, the total fraction of these oils is advantageously 0.5 to 15% by weight, based on the total weight of the cosmetic preparation. Paraffin oil and/or isoparaff in oil are preferably used in combination with the wax cera microcristallina, wherein the proportion by weight of paraffin oil and/or isoparaffin oil with respect to cera microcristallina is preferably from 1:1 to 1:3.

Furthermore, it is likewise advantageous in accordance with the invention, if the cosmetic preparation comprises one or more plant oils selected from the group evening primrose oil, rice bran oil, coconut oil, almond oil, walnut oil, peach kernel oil, apricot kernel oil, avocado oil, tea tree oil, soybean oil, sesame oil, sunflower oil, tsubaki oil, palm kernel oil, mango kernel oil, cuckoo flower oil, safflower oil, macadamia nut oil, grapeseed oil, amaranth seed oil, argan oil, bamboo oil, olive oil, wheatgerm oil, pumpkin seed oil, mallow oil, hazelnut oil, safflower oil, canola oil, sasanqua oil, liquid sheabutter oil (INCI: Butyrospermum parkii (Shea Butter) Oil), jojoba oil and rambutan oil.

If one or more plant oils are present, the total fraction thereof in the cosmetic preparation is advantageously 0.5 to 5% by weight.

By using the plant oils described above, after application of the preparation according to the specified method, the skin feels particularly soft. Also, a foam results which can be particularly easily distributed on the skin. After application, the skin tightens a little and the skin feels particularly well-groomed.

In addition, it is advantageous if the preparation according to the invention is characterized in that no particulate substances are present. It has been shown, advantageously, that in the absence of particulate substances, such as in Pickering emulsions for example, significantly more dimensionally stable foams are obtained. Moreover, the foams are perceived as better distributable on and washable off wet skin.

The cosmetic preparation according to the invention may advantageously comprise glycerol. It is preferable in accordance with the invention in this case if glycerol is present in a proportion from 3% by weight to 16% by weight, based on the total weight of the cosmetic preparation.

Further embodiments of the invention that are advantageous in accordance with the invention are characterized in that the cosmetic preparation according to the invention comprises phenoxyethanol, hydroxyacetophenone and/or ethylhexylglycerin for preservation.

If the cosmetic preparation comprises phenoxyethanol, it is advantageous according to the invention when the fraction of phenoxyethanol is from 0.1% by weight to 1% by weight, based on the total weight of the cosmetic preparation.

If the cosmetic preparation comprises hydroxyacetophenone, it is advantageous according to the invention when the fraction of hydroxyacetophenone is from 0.01% by weight to 0.5% by weight, based on the total weight of the cosmetic preparation.

If the cosmetic preparation comprises ethylhexylglycerin, it is advantageous according to the invention when the fraction of ethylhexylglycerin is from 0.1% by weight to 2% by weight, based on the total weight of the cosmetic preparation.

It is also advantageous in accordance with the invention if the cosmetic preparation comprises water in a proportion from 40% by weight to 80% by weight, preferably in a proportion from 50% by weight to 70% by weight, based on the total weight of the cosmetic preparation.

It is advantageous in accordance with the invention if the cosmetic preparation according to the invention comprises one or more active ingredients selected from the group of compounds gylcyrrhetic acid, urea, arctiin, alpha-lipoic acid, folic acid, phytoene, D-biotin, coenzyme Q10, hyaluronic acid, alpha-glucosylrutin, carnitine, carnosine, caffeine, natural and/or synthetic isoflavonoids, glycerylglucose, creatine, creatinine, taurine, β-alanine and/or licochalcone A, panthenol, tocopherol, tocopherol acetate, vitamin C, vitamin C derivative, Glycyrrhiza inflata root extract, magnolia extract, aloe vera.

The cosmetic preparation according to the invention is further advantageously characterized in that the cosmetic preparation has a viscosity in the range from 1000 mPa·s to 10 000 mPa·s, preferably 2000 mPa·s to 8000 mPa·s, and particularly preferably 1500 mPa·s to 5000 mPa·s at 25° C. It was particularly surprising that a preparation having such a high viscosity can be foamed and in this case, a particularly finely pored and structurally stable foam is obtained.

The viscosity of the cosmetic preparation according to the invention is determined as follows: measurement at 25° C. in a 150 ml rolled rim bottle using Rheomat R 123 from proRheo. The Rheomat R 123 from proRheo GmbH is a rotational viscometer, i.e. a measurement body rotates in the substance to be measured. The force is measured that is required to rotate the measurement body in the sample at a predefined speed. From this torque, the speed of the measurement body and the geometric dimensions of the measuring system used, the viscosity is calculated. The measurement body used is measurement body No. 1 (article no. 200 0191), suitable for a viscosity range of 500 to 9000 mPas, rotational speed range 62.5 min⁻¹.

It is also advantageous according to the invention if the cosmetic preparation has a pH in the range of 5 to 7.5.

For application, the cosmetic product according to the invention is firstly mixed thoroughly by shaking the pressure container and then withdrawn via the release valve and applied to the wet skin. On release from the pressure container, a foam or a foamed cosmetic preparation is obtained.

It is possible to use customary known aerosol can systems for cosmetic purposes as pressure containers with release valves. The pressure containers preferably have an inner riser tube which is attached to the release valve within the pressure container such that, on actuating the release valve, the preparation is withdrawn with the propellant from the pressure container via the riser tube and then through the release valve. Advantageously, the riser tube touches at least a part of the base of the pressure container.

Cosmetic preparations according to the invention having a viscosity in the range from 1000 mPa·s to 10 000 mPa·s, preferably 2000 mPa·s to 8000 mPa·s, and particularly preferably 1500 mPa·s to 5000 mPa·s at 25° C. could be foamed surprisingly well by using an aerosol can system comprising a pressure container with release valve and an inner riser tube. By using an inner riser tube, a surprisingly uniform foam could be obtained in the present viscosity range.

Pressure containers with spray or foam heads are preferably used. An example of a suitable foam head is the APTAR Accessories Foam Upright S25 Foam.

The cosmetic products of the present invention are advantageously produced in accordance with the invention as follows. The cosmetic preparation is obtained by means of the following steps:
(I) the polyacrylic acid polymers are dispersed in water and the resulting aqueous phase heated to 80° C.
(II) waxes and optionally further lipids, such as oils or fatty alcohols, are heated or melted at 80° C.
(III) both batches described above are mixed and homogenized at 80° C.
(IV) after cooling the batch to 25° C., further temperature-sensitive constituents such as perfumes or optionally preservatives can be added and the batch homogenized.

Subsequently, the cosmetic product is obtained in which the cosmetic preparation is filled into the pressure container together with propellants.

In the prior art, polyacrylic acid polymers are initially pre-dispersed in the usual way in a lipid or an oil, before these are dispersed in water. Due to the fact that the polyacrylic acid polymers, advantageously in accordance with the invention, are dispersed directly in water without lipids, increased deposition of lipids on wet skin is enabled on application of the preparation.

### COMPARATIVE EXPERIMENTS AND EXAMPLES

The examples below are intended to illustrate the present invention without limiting it. Unless otherwise stated, all quantitative data, fractions and percentages are based on the weight and the total amount or total weight of the cosmetic preparations.

| INCI | Ex. 1 | Ex. 2 | Ex. 3 | Ex. 4 | Ex. 5 |
|---|---|---|---|---|---|
| Cera Microcristallina | 13 | | 10 | | 5 |
| Paraffinum Liquidum | 12 | | 10 | | |
| Glycerol | 5 | 5 | 10 | 15 | 8 |
| Hydrogenated Coco-Glycerides | 3 | | 2 | | 5 |
| Acrylates/C10-30 Alkyl Acrylate Crosspolymer | 0.2 | 0.30 | 0.25 | 0.2 | 0.1 |
| Carbomer | 0.02 | 0.03 | | | |
| Myristyl Alcohol | 1 | 1 | | | |
| Cetearyl Alcohol | 5 | 4 | 3 | 3 | |
| Stearyl Alcohol | 2 | | 4 | | |
| Butyrospermum Parkii (Shea Butter) Oil | | 2 | | | |
| Hydrogenated Vegetable Oil | | 15 | | 12 | 5 |
| Prunus Amygdalus Dulcis Oil | 0.5 | 0.5 | | | |
| Simmondsia Chinensis (Jojoba) Seed Oil | | | | 3 | |
| Helianthus Annuus Seed Oil | | | | | 2 |
| Cocos Nucifera (Coconut) Oil | | | | | 1 |
| Cetyl Palmitate | | 3 | | 2.5 | |
| Aluminum Starch Octenylsuccinate | 1 | | | | |
| Perfume | 1 | 1 | 0.8 | 0.6 | |
| Sodium Hydroxide for pH adjustment | q.s. | q.s. | q.s. | q.s. | q.s. |
| Phenoxyethanol | 0.5 | 0.8 | | 0.3 | |
| Ethylhexylglycerin | | | 0.3 | 0.3 | 0.5 |
| Water | to 100 | to 100 | to 100 | to 100 | to 100 |
| pH | 7.0 | 6.5 | 7.2 | 6.8 | 7.0 |
| Filling in 200 ml can: | | | | | |
| Cosmetic preparation | 94% | 90% | 94% | 92% | 94% |
| Propellant fraction | 6% | 10% | 6% | 8% | 6% |
| Propellants | Propane/ | Propane/ | Propane/ | Propane/ | Propane/ |
| | butane/ | butane/ | butane/ | butane/ | butane/ |
| | isobutane | isobutane | isobutane | isobutane | isobutane |
| Pressure rating | 3.5 bar | 3.0 | 2.7 | 4.2 | 2.7 |
| Sensory properties of the foam | Creamy, | Creamy, | Creamy, | Creamy, | Creamy, |
| | stable, | stable, | stable, | stable, | stable, |
| | fine- | fine- | fine- | fine- | fine- |
| | bubble | bubble | bubble | bubble | bubble |
| | foam | foam | foam | foam | foam |
| Structural stability of the foam after 5 minutes | Stable | Stable | Stable | Stable | Stable |

The examples Ex. 1, Ex. 2, Ex. 3, Ex. 4 and Ex. 5 are inventive examples.

After application of the foam of example formula Ex. 1, this was stable under standard conditions for more than one hour and did not collapse in this time period.

1.-20. (canceled)

21. A cosmetic product, wherein the product comprises a pressure container having a release valve and containing
a) a cosmetic preparation which is free of surfactants and emulsifiers and comprises, based on a total weight of the preparation
(i) a total of 8% by weight to 35% by weight of one or more waxes which are different from fatty alcohols,
(ii) one or more polyacrylic acid polymers; and
b) one or more propellants which foam and/or discharge the cosmetic preparation.

22. The cosmetic product of claim 21, wherein the one or more waxes which are different from fatty alcohols are selected from one or more of cera microcristallina, hydrogenated jojoba oil, r hydrogenated vegetable oil.

23. The cosmetic product of claim 21, wherein a total fraction of polyacrylic acid polymers in the cosmetic preparation is from 0.02% to 2% by weight, based on the total weight of the cosmetic preparation.

24. The cosmetic product of claim 21, wherein the polyacrylic acid polymers have a viscosity as a 0.2 percent solution in water after neutralization of from 1,500 to 10,000 mPa·s at a temperature of 25° C.

25. The cosmetic product of claim 21, wherein the one or more polyacrylic acid polymers are polymerized from a monomer mixture comprising the monomers of the groups
(a.) acrylic acid and/or methacrylic acid;
(b.) C₁- to C₄₀-alkyl acrylates and/or C₁- to C₄₀-alkyl methacrylates;
and/or
(c.) ethylenically unsaturated hydrophobically modified monomers selected from polyethoxylated cetyl (meth)acrylate, polyethoxylated cetearyl (meth)acrylate, polyethoxylated stearyl (meth)acrylate, polyethoxylated arachidyl (meth)acrylate, polyethoxylated behenyl methacrylate, polyethoxylated cerotyl (meth)acrylate, polyethoxylated montanyl (meth)acrylate, polyethoxylated melissyl (meth)acrylate, polyethoxylated lacceryl (meth)acrylate, polyethoxylated nonylphenol (meth)acrylate, polyethoxylated tristyrylphenol (meth)acrylate, polyethoxylated palmyl itaconate, polyethoxylated stearyl itaconate, polyethoxylated behenyl itaconate, polyethoxylated palmyl maleate, polyethoxylated stearyl maleate, polyethoxylated behenyl maleate, polyethoxylated palmyl allyl ether, polyethoxylated stearyl allyl ether, polyethoxylated behenyl allyl ether, wherein a polyethoxylated portion of the above monomers comprises from 15 to 60 repeat ethylene oxide units;
with the proviso that the respective polyacrylic acid polymers of the aforementioned monomer groups (a.) to (c.) comprise either only the monomers of group (a.), only the monomers of the groups (a.) and (b.); or only the monomers of the groups (a.), (b.) and (c.).

26. The cosmetic product of claim 21, wherein the preparation comprises one or more polyacrylic acid polymers selected from carbomers and/or acrylates/C10-30 alkyl acrylate crosspolymers.

27. The cosmetic product of claim 21, wherein the one or more propellants comprise one or more of propane, butane, isobutane, pentane, isopentane, dimethyl ether, nitrogen, carbon dioxide.

28. The cosmetic product of claim 21, wherein the only propellants present therein are one or more of propane, butane, isobutane.

29. The cosmetic product of claim 21, wherein the pressure container contains from 90% by weight to 98% by weight of cosmetic preparation and 2% by weight to 10% by weight of the one or more propellants, based on a total weight of all liquids and gases present in the pressure container.

30. The cosmetic product of claim 21, wherein the cosmetic preparation has at least one melting range between 35° C. and 42° C. according to DSC.

31. The cosmetic product of claim 21, wherein the cosmetic preparation comprises one or more fatty alcohols having 6 to 22 carbon atoms at a total fraction of 0.1 to 15% by weight, based on the total weight of the cosmetic preparation.

32. The cosmetic product of claim 21, wherein the preparation further comprises one or more hydrocarbon oils selected from mineral oil, paraffin oil and isoparaffin oil.

33. The cosmetic product of claim 21, wherein the cosmetic preparation comprises cera microcristallina as wax different from fatty alcohols and paraffin oil and/or isoparaffin oil as hydrocarbon oil, a the proportion by weight of paraffin oil and/or isoparaffin oil with respect to cera microcristallina being from 1:1 to 1:3.

34. The cosmetic product of claim 21, wherein the cosmetic preparation comprises one or more plant oils selected from evening primrose oil, rice bran oil, coconut oil, almond oil, walnut oil, peach kernel oil, apricot kernel oil, avocado oil, tea tree oil, soybean oil, sesame oil, sunflower oil, tsubaki oil, palm kernel oil, mango kernel oil, cuckoo flower oil, safflower oil, macadamia nut oil, grapeseed oil, amaranth seed oil, argan oil, bamboo oil, olive oil, wheatgerm oil, pumpkin seed oil, mallow oil, hazelnut oil, safflower oil, canola oil, sasanqua oil, liquid sheabutter oil, jojoba oil, and rambutan oil.

35. The cosmetic product of claim 34, wherein a total fraction of plant oils in the cosmetic preparation is from 0.5% to 5% by weight, based on the total weight of the cosmetic preparation.

36. The cosmetic product of claim 21, wherein the cosmetic preparation has a viscosity in the range from 1000 mPa·s to 10 000 mPa·s, at 25° C.

37. The cosmetic product of claim 21, wherein the cosmetic preparation comprises from 9.5% to 25% by weight of the one or more waxes which are different from fatty alcohols.

38. A method of providing a foam which, on application to wet skin, deposits a lipid film on the skin and/or a method of providing a structurally stable foam, wherein the method comprises providing
(i) a total of 8% by weight to 35% by weight of one or more waxes which are different from fatty alcohols, and
(ii) one or more polyacrylic acid polymers
in a cosmetic preparation which is free of surfactants and emulsifiers and is foamed with propellant gas, the % by weight referring to a total weight of the cosmetic preparation without the propellant gas.

39. A method of applying the cosmetic product of claim 21, wherein the method comprises withdrawing the cosmetic preparation from the pressure container, applying the withdrawn cosmetic preparation to skin moistened with water, and rinsing the applied cosmetic preparation off the skin with water.

40. A method of applying the cosmetic product of claim 21, wherein the method comprises withdrawing the cosmetic preparation from the pressure container, applying the withdrawn foamed cosmetic preparation to hirsute skin moistened with water, and shaving hair in a skin region to which the cosmetic preparation has been applied with a razor blade.
